(19) United States
(12) Patent Application Publication
(10) Pub. No.: US 2008/0188863 A1
(43) Pub. Date: Aug. 7, 2008

(54) UTERINE ARTERY OCCLUSION

(76) Inventor: Michael S. H. Chu, Brookline, MA (US)

Correspondence Address: FAY KAPLUN & MARCIN, LLP, 150 BROADWAY, SUITE 702, NEW YORK, NY, 10038, US

(21) Appl. No.: 12/018,519

(22) Filed: Jan. 23, 2008

Related U.S. Application Data
(60) Provisional application No. 60/888,628, filed on Feb. 7, 2007.

(51) Int. Cl. A61B 17/42

(52) U.S. Cl. 606/119

(57) ABSTRACT

A system for occluding uterine arteries comprises a compression element shaped for insertion into the vagina to a target position in which a distal rim thereof engages a desired portion of tissue surrounding a cervical opening, the rim extending around a predetermined portion of a perimeter of the cervical opening and an anchoring mechanism locking a position of the compression element relative to the desired portion of tissue in combination with an advancing mechanism moving the compression element relative to the anchoring mechanism to compress the desired portion of tissue against an adjacent portion of a uterine wall capturing uterine arteries therebetween and occluding blood flow therethrough.

### PRIORITY CLAIM

This application claims the priority to the U.S. Provisional Patent Application Ser. No. 60/888,628, entitled “UTERINE ARTERY OCCLUSION,” filed Feb. 7, 2007. The specification of the above-identified application is incorporated herewith by reference.

### BACKGROUND

Conventional treatments for uterine fibroids have included drug therapies and hysterectomy. However, as drug therapies are often unsuccessful in more advanced cases and hysterectomy is an extreme measure, less invasive procedures such as arterial occlusion are often preferable as they tend to entail fewer and less severe side effects while reducing the duration of hospital stays and recovery periods.

### SUMMARY OF THE INVENTION

In one aspect, the present invention is directed to a system for occluding uterine arteries comprising a compression element shaped for insertion into the vagina to a target position in which a distal rim thereof engages a desired portion of tissue surrounding a cervical opening, the rim extending around a predetermined portion of a perimeter of the cervical opening and an anchoring mechanism locking a position of the compression element relative to the desired portion of tissue in combination with an advancing mechanism moving the compression element relative to the anchoring mechanism to compress the desired portion of tissue against an adjacent portion of a uterine wall capturing uterine arteries therebetween and occluding blood flow therethrough.

### BRIEF DESCRIPTION OF DRAWINGS

FIG. 1 is a side view of an uterus and cervix with the two uterine arteries;

FIG. 2 is a top, cross-sectional view of the uterus and cervix with the two uterine arteries;

FIG. 3 is a diagram showing a first embodiment of an uterine artery occlusion device according to the invention;

FIG. 4 is a diagram showing a second embodiment of the uterine artery occlusion device according to the invention;

FIG. 5 is a perspective view of the device of FIG. 4;

FIG. 6 is a diagram showing an elastic band deployed (e.g. by the occlusion device of FIG. 4);

FIG. 7 is a diagram showing a third embodiment of the uterine artery occlusion device according to the invention;

FIG. 8 is a perspective view of a fourth embodiment of the uterine artery occlusion device according to the invention;

FIG. 9 is a diagram showing a fifth embodiment of the uterine artery occlusion device according to the invention;

FIG. 10 is a diagram showing a sixth embodiment of the uterine artery occlusion device according to the invention;

FIG. 11 is a depiction showing an embodiment of a detachable global fibroid occlusion cup according to the invention;

FIG. 12 is a depiction showing a second embodiment of a detachable global fibroid occlusion cup according to the invention;

FIG. 13 is a depiction showing a third embodiment of the global fibroid occlusion cup according to the invention;

FIG. 14 is a depiction showing a fourth embodiment of the global fibroid occlusion cup according to the invention;

FIG. 15 is a depiction showing a fifth embodiment of the global fibroid occlusion cup according to the invention;

FIG. 16 is a depiction showing a sixth embodiment of the global fibroid occlusion cup according to the invention;

FIG. 17 is a depiction showing a seventh embodiment of the global fibroid occlusion cup according to the invention; and

FIG. 18 is a perspective view of a further embodiment of a global fibroid occlusion cup with a balloon anchoring mechanism according to the invention.

### DETAILED DESCRIPTION

The present invention may be further understood with reference to the following description and to the appended drawings, wherein like elements are referred to with the same reference numerals. The present invention relates to devices for treatment of uterine diseases by occlusion of the uterine arteries. In particular, the invention relates to less invasive methods and systems for occluding the uterine arteries.

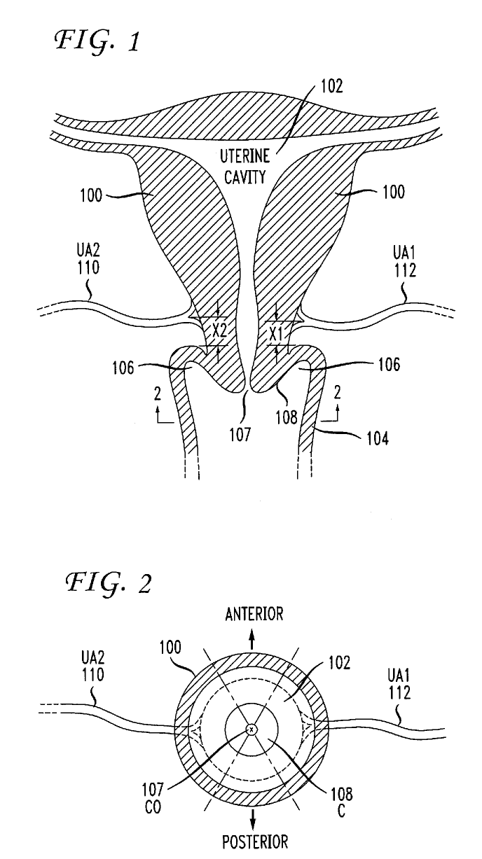

Fibroids have been effectively treated by occluding the blood supply from the two uterine arteries feeding the uterus. Human uterine arteries are typically located about 3 cm or less from the vaginal wall at the vaginal formix, where the uterine artery meets the uterus. It is thus possible to pinch the arteries and occlude the flow of blood by pressing on the vaginal formix 106 from inside the vagina 104. FIGS. 1 and 2 show the uterus 100, the vagina 104 and the vaginal formix 106 in their relative positions.

FIG. 1 shows the path from the vagina 104 to the uterine cavity 102 via the cervix 108 and the proximal cervical os 107 with the vaginal formix 106 surrounding the proximal cervical os 107 and slightly distal thereto. That is, the cervical os 107 protrudes slightly into the vagina 104 so that the vagina 104 is slightly deeper at the vaginal formix 106—an annular area surrounding the cervical os 107. The uterine arteries 110, 112 extend to the walls of the uterus 100 from the inferior iliac artery (not shown) and are located generally symmetrically about the uterus 100. As seen in FIG. 2, when viewed from the above with the 12 o'clock position directly forward, the right uterine artery 112 is typically located between the 1 and 5 o'clock positions, while the left uterine artery 110 is typically between the 7 and 11 o'clock positions.

Conventional occlusion procedures require accurate location of each of the arteries 110, 112 using, for example, doppler or other audio and imaging techniques that are difficult and time consuming and which require highly trained operators. Devices and methods according to embodiments of the present invention simplify occlusion procedures by reducing or eliminating the need to accurately locate the arteries 110, 112.

Devices and methods according to exemplary embodiments of the invention non-invasively occlude the flow of blood through all arteries in an arc of up to 360E around the uterus 100 lowering the level of skill necessary to successfully identify and occlude each of the arteries 110, 112.

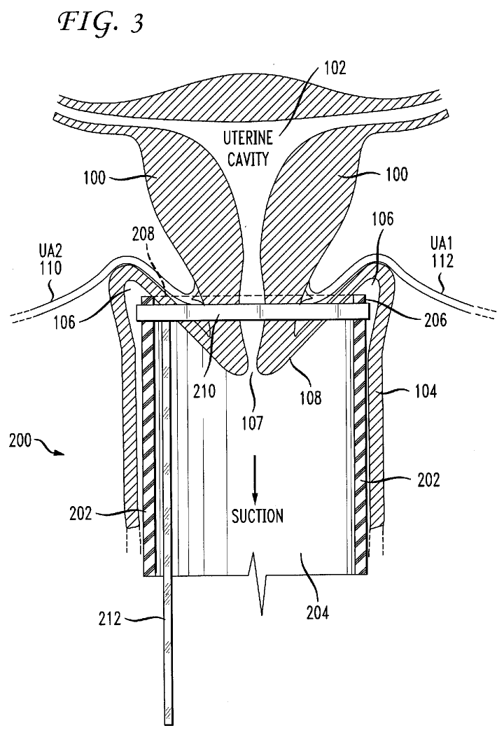

As shown in FIG. 3, an occlusion device 200 according to the invention is sized and shaped for placement within the vagina 104, allowing the patient freedom of movement during the occlusion procedure without the risk of inadvertent release of the device 200. The device 200 comprises a tube 202 sized and shaped for insertion into the vagina 104. The tube 202 has an open distal end 206 with a distal rim 208 which, as the tube 202 is inserted into the vagina 104, engages the vaginal formix 106 surrounding the cervical os 107. The tube 202 also defines a lumen 204 which provides access to the cervix 108 via the proximal cervical os 107. An inner diameter of the open distal end 206 is selected to match an external diameter of a proximal end of the wall of the uterus 100. An anchoring mechanism is then utilized to draw the uterus 100 and the open distal end 206 of the tube 202 toward the uterus 100, and to maintain it in place during the procedure. In this exemplary embodiment, the anchoring mechanism comprises a vacuum device (not shown) that applies suction to the lumen 204 so that the cervix 108 and the proximal end of the uterus 100 are drawn into the lumen 204 moving proximally relative to the formix 106 by 4 cm or more while the rim 208 is forced against the formix 106. As shown in FIG. 3, as the cervix 108 and the proximal end of the uterus 100 are drawn proximally relative to the formix 106, the uterine arteries 110, 112 are pinched between the external surfaces of the formix 106 and the uterus 100, occluding blood flow therethrough. Those skilled in the art will understand that graduated markings may be placed on the tube 202 to indicate a depth of insertion of the tube 202 into the uterus 100. In addition, as would be understood by those skilled in the art, the tube 202 may optionally be made of a transparent material or have transparent windows formed on its surface to provide visual guidance ensuring accurate placement and visual monitoring of the progress of the procedure.

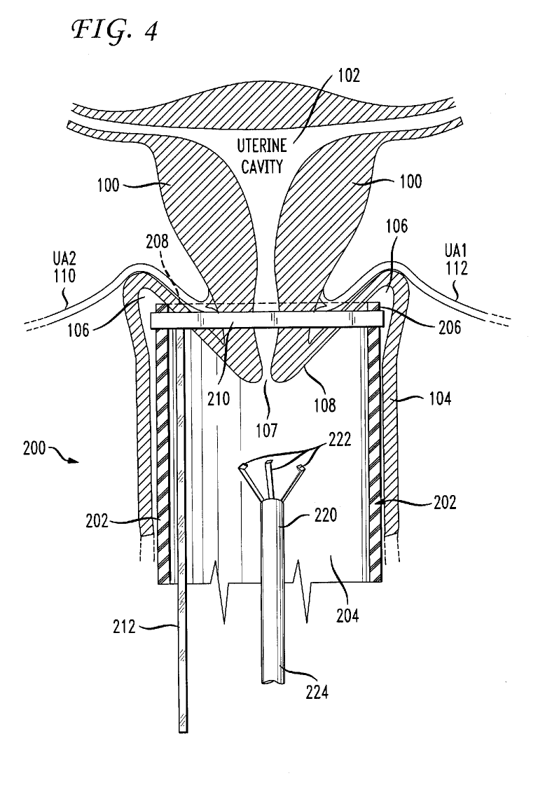

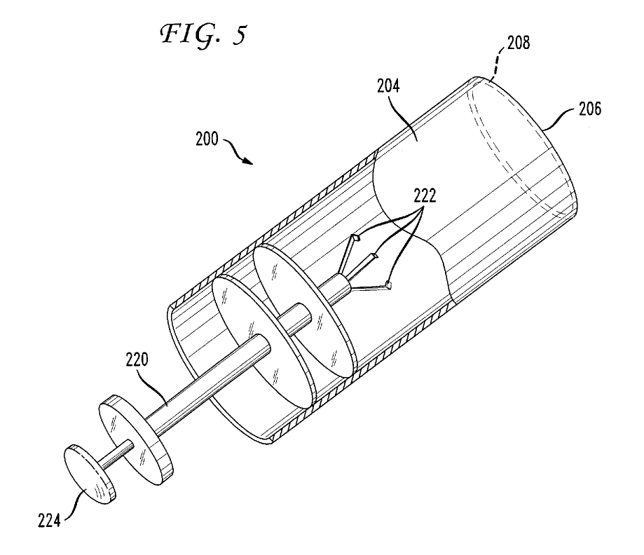

In a different embodiment shown in FIGS. 4 and 5, the anchoring mechanism of the device 200 comprises forceps 220 used to draw the cervix 108 proximally into the lumen 204. As would be understood by those skilled in the art, fingers 222 of the forceps 220 grasp tissue surrounding the cervical os 107 and retract this tissue proximally toward the vagina 104 through the distal opening 206 while the rim 208 is maintained stationary against the formix 106. Thus, the cervix 108 and the proximal end of the uterus 100 are drawn into the tube 202 in a manner similar to that described above. The forceps 220 according to this embodiment include an elongated shaft 224 permitting operation and manipulation from outside the body.

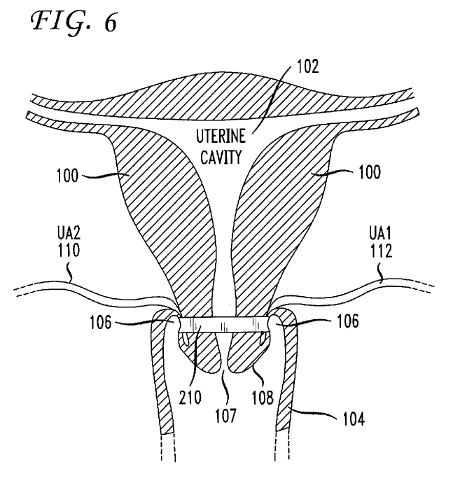

The embodiments shown in FIGS. 3-5 comprise an elastic band 210 mounted at the distal end of the tube 202 adjacent the rim 208. The elastic band 210 is stretched around the tube 202 adjacent to the distal end 206 thereof and is coupled to a deployment member 212 which may be a filament (e.g., wire looped around the elastic band) or a push off member having sufficient rigidity that it may push the elastic band 210 distally off of the distal end 206 of the tube 202. That is, after the proximal end of the uterus 100 and the cervix 108 have been advanced proximally into the distal end of the tube 202, the elastic band 210 is deployed by drawing the deployment member 212 proximally relative to the tube 202 (if the member 212 is a wire) or by pushing the member 212 distally (where the member 212 is a rigid push off member) until the elastic band 210 is released from the distal end of the tube 202. As shown in FIG. 6, the elastic band 210 then contracts around the tissue which had been drawn into the tube 202 (e.g., the vaginal wall adjacent to the cervical os 107 and the formix 106 along with the cervix 108 and the proximal end of the uterus 100). The elastic band 210 is preferably sized so that, when released from the tube 202, it contracts to a significantly reduced diameter applying a desired compressive force around an entire circumference of the proximal portion of the uterus pinching the uterine arteries 110, 112 between the vaginal formix 106 and the uterus 100 and occluding blood flow through the arteries 110, 112 regardless of their location around the circumference of the uterus 100. For example, the elastic band 210 may apply 10 to 20 pounds of compressive force to the tissue received therein and may be surface treated to prevent the band 210 from rolling off of the formix 106. The delivery portion of the device (including the tube 202 and the vacuum mechanism or forceps 220) is then withdrawn and the elastic band 210 remains in place to occlude flow through the uterine arteries 110, 112 on its own. As would be understood by those skilled in the art, the band 210 may be composed of a material selected to degrade after a predetermined time has elapsed or may be snipped and removed after the desired time has elapsed.

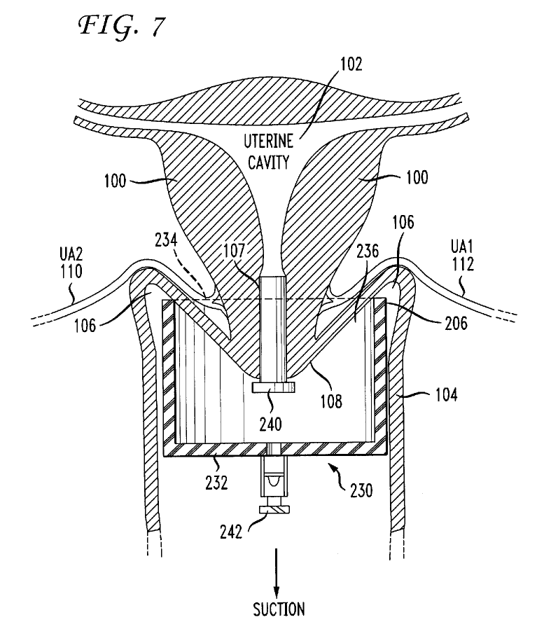

As shown in FIG. 7, an occlusion device 230 according to a further embodiment of the invention includes a cup 232 which substantially corresponds to the tube 202 and which is seated against the vaginal formix 106 as in the above described embodiments. As described in more detail below, the device 230 further includes a plug 240 which is inserted into the cervix 108 to seal and stiffen the cervix 108 and to provide a more substantial and rigid surface against which to compress the proximal end of the uterus 100 and the uterine arteries 110, 112. As would be understood by those skilled in the art, suction may be applied to the cup 232 via any conventional fluid connection such as a one way valve 242.

As described above, the cup 232 is inserted into the vagina 104 with the open end 236 facing distally to receive the tissue surrounding the cervical os 107 in an open distal end thereof with a rim 234 seated in the vaginal formix 106. Similarly to the above described embodiments, as suction is applied to the cup 232, the tissue surrounding the cervix 108 along with the proximal portion of the uterus 100 is drawn about 3 to 4 cm into the cup 232 pressing the rim 234 against the vaginal formix 106 and pressing the formix 106 over the uterine arteries 110, 112 and against the external surface of the uterus 100. The plug 240 reduces the compression of the uterus 100 enhancing the pinching off of the arteries 110, 112 facilitating their occlusion. To maintain the occlusion of these vessels for a desired time, suction is applied to the cup 232 for the duration of the procedure using, for example, a one way valve 242 which may, as would be understood by those skilled in the art, include a fitting for a vacuum line or other connection to a source of negative pressure. In this embodiment, the plug 240 is inserted into the cervix 108 before the cup 232 is introduced into the vagina 104. After the procedure has been completed, usually lasting about 6 hours, the vacuum is released (e.g., by opening the one way valve 242) and the cup 232 is removed. The plug 240 may then be removed as well.

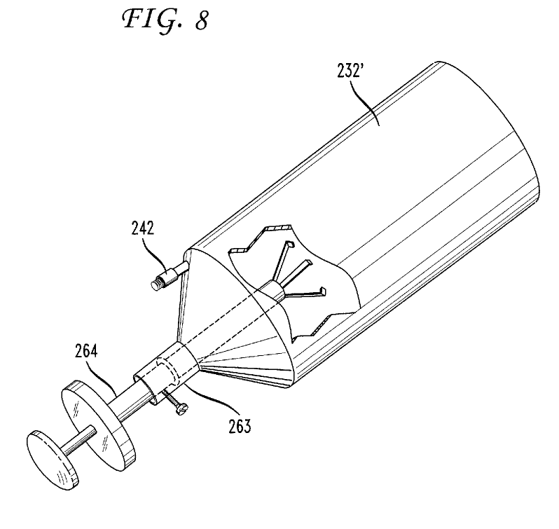

The device 230 is described as employing suction only to drawn the cervix 108 and the proximal end of the uterus 100 into the cup 232 to pinch the arteries 110, 112. However, as shown in FIG. 8, a cup 232′ allows an operator to use the device in conjunction with a forceps or suction alone or with a forceps in conjunction with suction. The cup 232′ is substantially similar to the cup 232 of FIG. 7 except that it includes a forceps access port 263 through which a forceps 264 may be inserted while maintaining an airtight seal within the cup 232′.

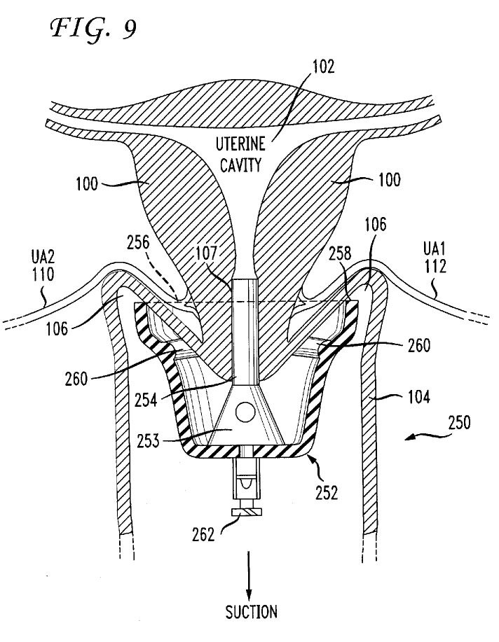

In a different exemplary embodiment, the stiffening plug and the cup device may be formed as a single piece. As shown in FIG. 9, the occlusion device 250 comprises a cup 252 with an opening 256 that is placed around the cervical os 107 so that the rim 258 seats against the tissue of the vaginal formix 106. An optional ridge 260 protrudes from the inside surface of the cup 252, near the rim 258 to focus pressure and enhance the pinching off of the arteries 110, 112 by more firmly pressing the tissue of the formix 106 against the proximal end of the uterus 100. For example, the ridge 260 may extend 360 degrees around the cup 252, or may be placed only at selected locations such as in the 1 to 5 o'clock position and the 7 to 11 o'clock position where the arteries 110, 112 are typically located. Thus an increased pressure is applied to areas within which the arteries 110, 112 are likely located.

The plug 254 is coupled to the cup 252 via a connecting member 253 extending distally from a proximal end of the cup 252 to couple to a proximal end of the plug 254. In one embodiment, the connecting member 253 is substantially rigid so that the plug 254 cannot move relative to the cup 252. In addition, as would be understood by those skilled in the art, the plug 254 may, optionally, be integrally formed with the cup 252 obviating the need for a separate connecting member 253.

The device 270 shown in FIG. 10 is substantially similar to the device 250 except that, instead of the valve 262, the proximal end of the cup 272 includes a port through which a forceps 280 is introduced into the interior space of the cup 272 to draw tissue into the cup 272 as described above. The cup 272 fits over the formix 106 and has a rim 274 to compress the formix 106 and pinch the uterine arteries 110, 112 as described in regard to the above embodiments. As with the forceps described above, the forceps 280 comprise arms 282 which grasp the tissue surrounding the cervix 108 and a shaft 278 allowing for remote manipulation and positioning of the forceps 280. A motion element is provided to translate the cup 272 along the shaft 278. For example, the shaft 278 may have threads 284 that cooperate with threads 286 on the cup 272 so that rotation of the shaft 278 relative to the cup 272 in a first direction generates proximal movement of the shaft relative to the cup 272 while rotation in the opposite direction moves the shaft distally relative to the cup 272. Alternatively as would be understood by those skilled in the art, other mechanisms may be used to move the cup 272 relative to the shaft 278. For example, a spring mechanism may be used to bias the two components in a desired direction and to advance the cup 272 distally against the formix 106.

Additional embodiments of the present invention may be devised to advance a compression device such as a cup against the vaginal formix to occlude the flow of blood to the uterus. Because the occlusion surface according to the invention extends substantially 360E around the uterus, there is no need to accurately locate the arteries, and all possible branchings of the arteries are encompassed without having to angularly orient the rim of the device. To further simplify the procedure, mechanical means are provided to facilitate advancement of the compression element over the anchoring mechanism after the device has been inserted into the vagina. For example, single hand placement and advancement of the device is possible using the embodiments of the invention.

As shown in FIG. 13, an exemplary uterine artery occlusion instrument 300 comprises an elongated cylindrical barrel 302 having a distal end with a rim 310 formed by the entire leading edge of the barrel 302. However, those skilled in the art will understand that the rim 310 may extend only along a portion or portions of the leading edge of the barrel 302 which, when in a desired orientation, are likely to engage portions of tissue aligned with the uterine arteries. The barrel 302 is preferably made of a transparent material or, alternatively, may include windows to provide viewing thereinto. In one embodiment, the rim 310 has a diameter of about 1.25 inches, to fit over and occlude the arteries of most patients. However, those skilled in the art will understand that the diameter and/or the shape of the rim 310 may be altered as desired to fit the anatomy.

The barrel 302 guides the entire device 300 as it is inserted into the vagina and aligns a center shaft 306 extending therein with the opening to the cervix. A distal tip 312 of the center shaft 306 is inserted into the cervical opening and the anchoring means is used to secure the device 300 in place as described above. For example, external threads 308 may be provided near the distal end of the center shaft 306 to anchor the device by threading the center shaft 306 into the cervical canal. The barrel 302, a proximal end of which is attached to a handle portion 304 for manipulating the device 300, contains controls to advance the barrel 302 distally relative to the center shaft 306 and to retract the barrel 302 proximally relative thereto.

In one exemplary embodiment, the advancing mechanism comprises a trigger 320 that is manually squeezed against a grip 322 to advance the barrel 302 relative to the threads 308 of the anchoring portion. Thus the barrel 302 and the rim 310 are manually advanced linearly and rapidly by simply squeezing the trigger 320 to engage the tissue surrounding the cervical opening and occlude the uterine arteries as described above. As would be understood by those skilled in the art, any of a variety of conventional mechanical linkages may be employed to transform the movement of the trigger 320 to distal and proximal motion of the barrel 302 relative to the center shaft 306. An angled plate 326 within the trigger mechanism prevents proximal movement of the barrel 302 as it is advanced until such time as the barrel 302 is unlocked for proximal withdrawal.

Markings may be provided on the barrel 302 and/or on the shaft 306 to indicate a depth of insertion of the rim 310, which in most cases will be about 3 to 4 inches. Doppler measurements or other sensors may be used to measure the depth of insertion or alternatively the start/end positions of the rim 310 relative to the threads 308 may be measured.

Typically the occlusion procedure is maintained for a period of time sufficient to ensure that the fibroids have necrosed to a desired degree while allowing the uterus to fully recover after blood flow has been restored. Those skilled in the art will understand that this may require occlusion of blood flow for 6 hours or more. Once the desired duration of occlusion has elapsed, the barrel 302 is withdrawn to release the compression of the uterine arteries by pressing a smaller trigger 324 that releases the angled plate 326 allowing proximal movement of the barrel 302 over the center shaft 306.

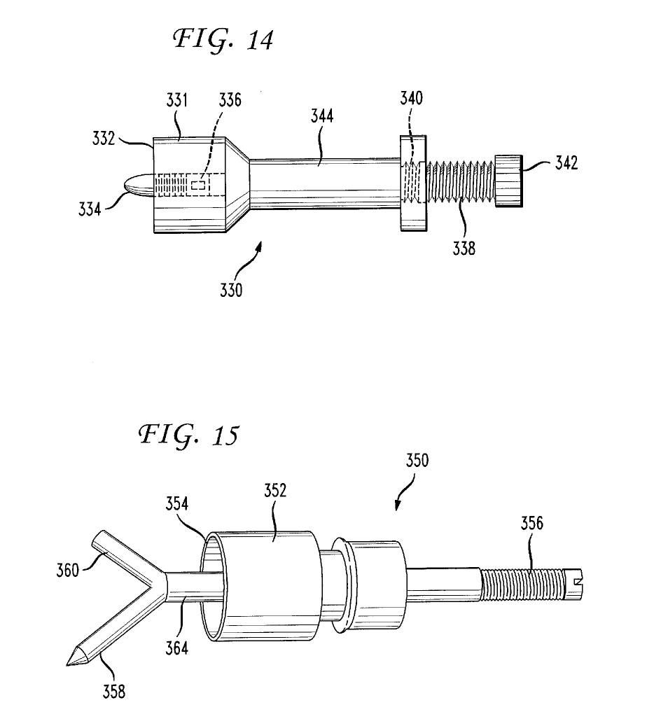

As shown in FIG. 14, a global fibroid occlusion instrument 330 according a further embodiment of the invention comprises a cup 331 having a rim surface 332. The cup 331 is dimensioned to fit around the cervix receiving a proximal portion of the uterus as described above with the rim surface 332 pressing the formix against the uterine arteries. A deployment element comprising a center shaft 334 penetrates the cervical opening with external anchoring threads 336 forming an anchor which, when engaged within the cervix, retains the instrument 330 in a desired position within the vagina. As described above in regard to FIG. 13, the cup 331 may be formed of a transparent material, or may have window-like openings to permit observation of the center shaft 334.

The center shaft 334 comprises rear threads 338 forming an advancing mechanism for the cup 331 together with corresponding threads 340 linked to the cup 331. The center shaft 334 may be turned, for example using the knob 342, to advance and retract the rim 332 relative to the anchoring mechanism formed by the anchoring threads 336. The cup 331 may be operatively connected to the threads 340 through an extension tube 344, or may be an integral part thereof.

During use, the cup 331 can be advanced over the front anchoring screw thread 336 by rotating the knob 342 clockwise, to protect the vaginal walls. Before inserting the anchor thread 336 into the cervical canal the cup 331 is bottomed on the center shaft 334 by rotating it counterclockwise to its stops. The center shaft 334 is then placed in the cervical canal so that the entire device is anchored by rotating the knob 342. The cup 331 is then advanced distally against the formix by rotating it over the threads 338 while the center shaft 334 is anchored into the cervical canal via the threads 336. The cup 331 is further advanced by turning the knob 342 if needed, so that the rim 332 compresses and occludes the uterine arteries.

As shown in FIG. 15, an instrument 350 according to another embodiment of the invention comprises a cup 352 with a rim 354 that forms a compression element for occluding the uterine arteries substantially as described above. The cup 352 is advanced over a center shaft 364 using an advancing mechanism such as threads 356 which mate with corresponding threads (not shown) on an interior of a lumen of a shaft of the instrument 350. The anchoring mechanism comprises a pair of substantially coaxial curved shafts 358, 360 which, in an insertion configuration, are entirely contained within a lumen of the center shaft 364.

Once the center shaft 364 has been extended to extend its distal end through the cervical canal into the uterus, the internal shaft 358 is advanced distally and rotated 180E relative to the external shaft 360 to form a Y shaped anchor. The deployed diameter of the Y shaped anchor is preferably less than a diameter of the cup 352, so that the rim 354 can advance distally beyond the anchor. The Y shaped anchor formed by the internal and external shafts 358, 360 is less invasive and stronger than other types of anchor, because it does not thread or puncture the tissue of the cervix. As would be understood by those of skill in the art, an advancing mechanism comprising threads, a linear drive or other elements may be used to advance the cup 354. In addition, a locking device similar to any of those described above in regard to the other embodiments of the invention may be employed to assist in maintaining the cup 354 in a desired position during the procedure.

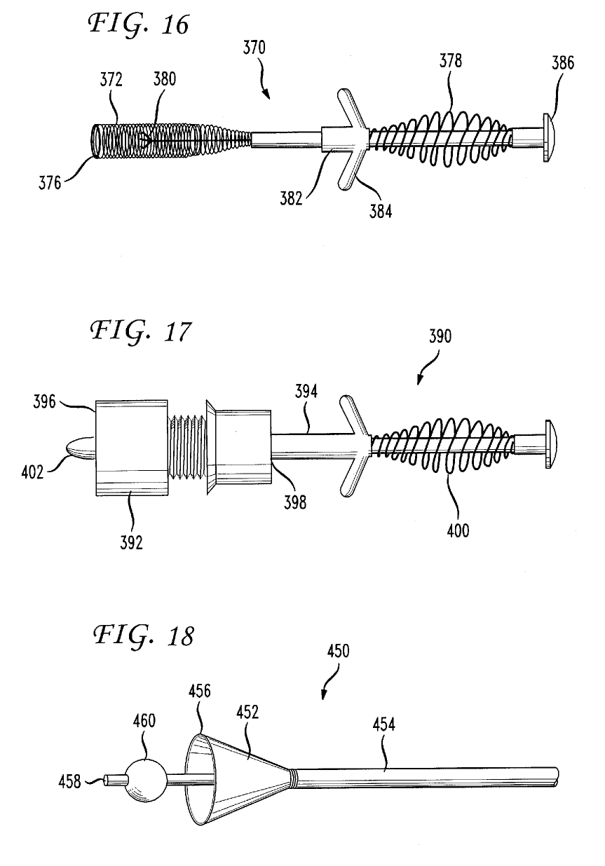

As shown in FIG. 16, an instrument 370 according to a further embodiment of the invention comprises a rigid coil 372 defining a distal rim 376. As would be understood by those skilled in the art, the coil 372 which may be welded to form a closed loop for this application acts as a screw enabling the rim 376 to be advanced distally relative to an anchoring mechanism. For example, the rim 376 may be advanced distally and withdrawn relative to a forceps 380 located within the coil 372 by rotating the forceps 380 relative to the coil 372. A handle portion 382 is provided for manual operation and positioning of the device by a user. The grips 384, 386 may be moved to open and close arms of the forceps 380 and may be rotated to advance the coil 372 relative to the rim 376. A coil 378 may optionally be included to bias the forceps 380 toward a closed or gripping position so that, once positioned over target tissue, the forceps 380 grip tissue without continued gripping by the user.

As shown in FIG. 17, an instrument 390 according to a related exemplary embodiment comprises a polymer cup 392 defining a rim 396 which is advanced over a center shaft 394 using an advancing mechanism 398, as described above. A forceps 402 extends coaxially through the center shaft 394 and is controlled, for example, by a handle portion 400 which, during the procedure, remains available outside the body. As described above, the forceps 402 may be biased toward the closed position by, for example, a spring.

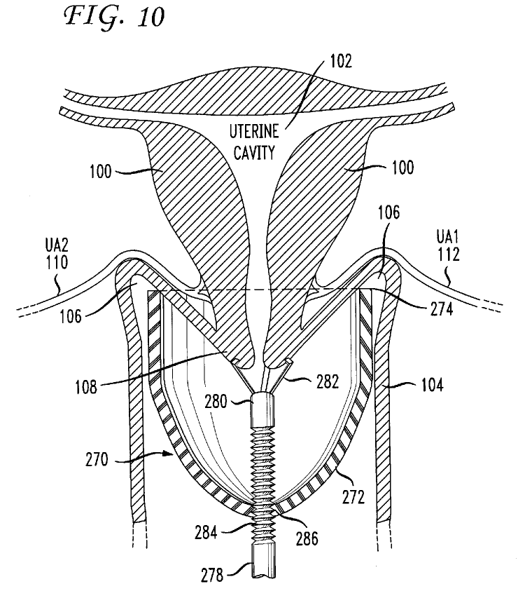

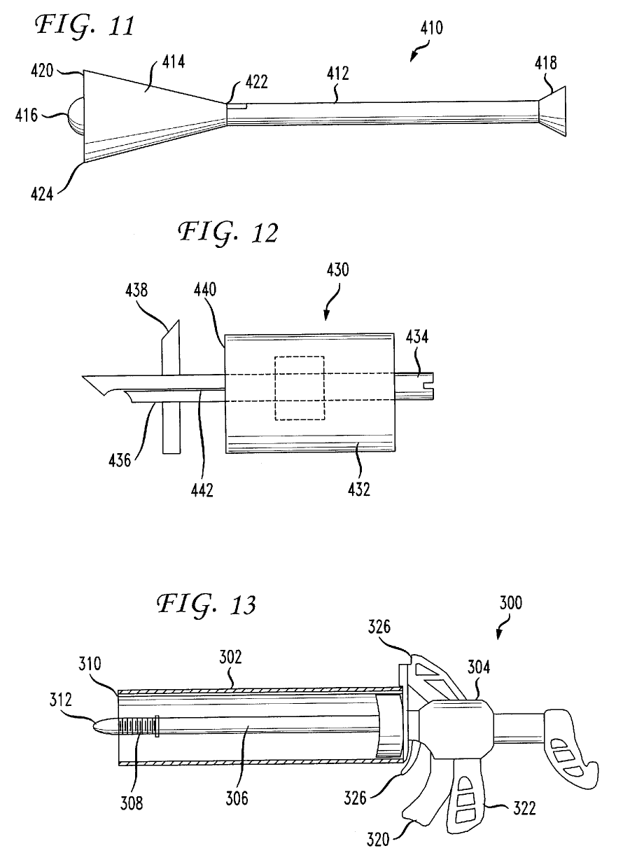

The occlusion instruments described above comprise a compression element such as a cup with a rim that may remain attached to the delivery mechanism. However, it may be beneficial to detach the compression element from the delivery portion, so that the patient may be more mobile and have less discomfort during the procedure. As shown in FIG. 11, an instrument 410 according to a further embodiment of the invention comprises an elongated center shaft 412 with a manipulating handle 418. A detachable fibroid cup 414 defining a distal rim 420 is connected to the center shaft 412 by means of a releasable connection 422. For example, the releasable connection 422 may comprise a backstop preventing the fibroid cup 414 from translating proximally, and a keyway for transmitting torque from the handle 418 to the cup 414.

In this exemplary embodiment, the entire leading edge of the detachable cup 414 forms the rim 420. The detachable cup 414 may be made of a transparent or translucent material or may comprise windows designed to enable the user to visually operate the device. The center shaft 412 may include an anchoring mechanism such as, for example, external threads 416 which grip inner walls of the cervical canal and, when rotated draw the cup 414 further distally into the vagina compressing the formix and, eventually, occluding the uterine arteries as described above. The detachable cup 414 further comprises internal threads 424 grasping outer walls of the cervix and the formix to further anchor the cup 414 in place.

The entire device 410 is inserted into the vagina such until a distal end of the center shaft 412 enters the cervical canal. The shaft 412 is then rotated using the handle 418 so that the threads of the detachable cup 414 engage the cervix and draw the center shaft 412 and the cup 414 distally into the vagina. The user continues to rotate the shaft 412 until the rim 420 engages the formix and compresses it against the proximal portion of the uterus occluding the uterine arteries as described above. As would be understood by those skilled in the art, the depth of advancement of the detachable cup 414 may be monitored using a doppler system, other sensors, or by measurement marks on the center shaft 412 as described above to ensure that the cup 414 reaches a desired position and is not inserted distally beyond a safe distance.

Once the detachable cup 414 is in place, the center shaft 412 is released and removed from the body, leaving the cup 414 in place to continue occluding the uterine arteries. This allows the patient to be mobile during the procedure. After the desired occlusion period has elapsed (e.g., 6 hours), the shaft 412 is reinserted and attached to the detachable cup 414. Then the shaft 412 is rotated in the opposite direction to remove the cup 414 from the body.

As shown in FIG. 12, an instrument 430 according to another embodiment of the invention comprises a detachable cup 432 with a rim surface 440 and a center shaft 436. Anchoring means are provided in the form of a toggle 438 which is housed in the center shaft 436 during insertion. In use, the instrument 430 advanced to a desired position by inserting the center shaft 436 through the cervical canal into the uterus. At this point, a screw 434 is rotated (e.g., using a screw driver or other device) to advance against the toggle 438 biasing the toggle 438 outward toward the open position shown in FIG. 12 to anchor the shaft 436 in the proximal portion of the uterus.

Continuing to rotate the screw 434 causes the threads 442 of the toggle 438 to engage the screw 434, and to pull the toggle 438 proximally toward the rim 440. This causes the toggle 438 to engage tissue at the distal opening of the cervical canal into the uterus while drawing the rim 440 distally against the formix compressing the vaginal wall and the proximal portion of the uterine wall to occlude the uterine arteries as described above. The correct depth of the detachable cup 432 may be measured as described above, with sensors or with markings. When the rim 440 has reached a desired position relative to the toggle 438, the device for rotation of the screw 434 is removed, leaving the detachable cup 432 and the short center shaft 436 in place until the desired occlusion time has elapsed.

In one embodiment, the detachable cup 432 is not threaded to the screw 434, but uses a linear advancing mechanism. The detachable cup 432 thus travels linearly without rotating as it is pulled towards the toggle 438. As in previous embodiments, the detachable cup 432 may be transparent or may have windows build into its surfaces. The anchoring mechanism that comprises the toggle 438 provides a more secure mechanical clamping force than is possible to obtain by threading a screw in the tissue, and may result in less damage to the cervical walls.

A different anchoring mechanism for an occlusion instrument according to the invention is shown in FIG. 18. The occlusion instrument 450 comprises a center shaft 454 with a cup 452 having a rim 456 defining the compression element. A balloon member 460 is disposed near the distal end 458 of the center shaft 454 to anchor the device in the uterus. The distal end 458 of the center shaft 454 is inserted into the uterus through the cervix and the balloon member 460 is inflated within the uterus. An advancing mechanism as described above may be used to advance the rim 456 towards the balloon element 460, and to force the rim 456 against the vaginal formix and to occlude the uterine arteries. As would be understood by those skilled in the art, any of the anchoring mechanisms may be combined with any of the various advancing mechanisms and/or with any of the cup/rim arrangements described herein as desired.

In another embodiment, the rim 456 may be stationary relative to the center shaft 454. In this embodiment, the balloon element 460 is shaped such that as it inflates, the distance between the balloon 460 and the cup 452 is reduced to the point that the rim 456 is forced against the vaginal formix pushing the proximal end of the uterus against the formix to occlude the uterine arteries pinched therebetween. For example, the balloon element 460 may be shaped to fit the contours of the lower portion of the uterus, just above the opening of the cervical canal. The mechanical advancement of the cup 452 and the force due to the inflation of the balloon element 460 may be combined to achieve additional clamping force on the arteries.

The inflation mechanism of the balloon element 460 may comprise an inflation tube 462 that is connected to a source of fluid, such as air, saline etc. In another embodiment, the balloon element 460 may comprise a thick walled elastic element, for example a tubular element that expands radially when compressed axially along the length of the center shaft 454. A conventional mechanism to compress the elastic element may be operable by the user. Alternatively, the balloon element 460 may be replaced with a solid dilator shaped to resemble an inflated balloon.

Those of skill in the art will understand that the shape of the compression element according to the present invention may be modified to suit specific applications. For example, the shape of the cup and of the rim may be varied according to the invention. The shape of the rim may be circular, oval, or other shapes that fit over the vaginal formix and permit application of an occlusive force to the uterine arteries. The surface of the rim may have different textures and elevations, as necessary to apply the occlusive force and to reduce injury to the tissue. Likewise, the rim surface may have a shape other than a cup or barrel, and does not have to form a closed circle. For example, the rim may include two wings oriented at 180E relative to one another with each wing subtending a range of at least 120E. When, in certain cases, it is desired to apply an evenly distributed force, the surface of the rim is preferably substantially coaxial with the center shaft, symmetric to the centerline. However, other arrangements are possible (e.g., to apply compressive force asymmetrically) and the term “center shaft” is not intended to imply that this shaft is required to extend along a central axis of the cup or barrel.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. A system for occluding uterine arteries, comprising:
a compression element shaped for insertion into the vagina to a target position in which a distal rim thereof engages a desired portion of tissue surrounding a cervical opening, the rim extending around a predetermined portion of a perimeter of the cervical opening;
an anchoring mechanism locking a position of the compression element relative to the desired portion of tissue; and
an advancing mechanism moving the compression element relative to the anchoring mechanism to compress the desired portion of tissue against an adjacent portion of a uterine wall capturing uterine arteries therebetween and occluding blood flow therethrough.

2. The system according to claim 1, further comprising:
a stiffening element inserted into the cervical canal to enhance compression of the desired portion of tissue and the corresponding portion of the uterine wall thereagainst.

3. The system according to claim 1, wherein the rim extends around at least first and second radially opposed portions, each of the first and second portions subtending an angle of at least 120E.

4. The system according to claim 1, wherein the rim extends substantially around an entire perimeter of the cervical opening.

5. The system according to claim 1, further comprising:
a deployment device coupled the anchoring mechanism and the compression element so that, after the anchoring mechanism and the compression element have been moved relative to one another to compress the desired portion of tissue against the adjacent portion of the uterine wall to occlude the uterine arteries, the deployment device may be detached therefrom and withdrawn from the body leaving the compression element and the anchoring mechanism in place.

6. The system according to claim 1, wherein the compression element comprises a cup having an open, substantially circular distal end forming the rim.

7. The system according to claim 6, wherein the cup is substantially cylindrical.

8. The system according to claim 6, wherein the cup is substantially conical.

9. The system according to claim 1, wherein the compression element comprises a deployment carrier and an elastic band deployable from the deployment carrier.

10. The system according to claim 1 wherein the compression element comprises a coil.

11. The system according to claim 10, wherein the rim is formed at an open distal end of the coil.

12. The system according to claim 2, wherein the stiffening element comprises a shaft of the anchoring mechanism.

13. The system according to claim 1, wherein the anchoring mechanism comprises a forceps.

14. The system according to claim 1, wherein the anchoring mechanism comprises a shaft with external threads which, when in an operative position, engage an inner wall of the cervix.

15. The system according to claim 1, wherein the anchoring mechanism comprises a coil.

16. The system according to claim 1, wherein the anchoring mechanism comprises a vacuum lumen connectable to a source of negative pressure to draw tissue into the compression element.

17. The system according to claim 16, wherein the vacuum lumen includes a one way valve maintaining the negative pressure within the compression element.

18. The system according to claim 1, wherein the anchoring mechanism comprises a shaft slidable through the compression element into the uterus, the shaft being deployable to engage tissue.

19. The system according to claim 1, wherein the anchoring mechanism comprises a flexible element expandable within the uterus to a diameter greater than that of a cervical opening.

20. The system according to claim 19, wherein the flexible element comprises an inflatable balloon.

21. The system according to claim 19, wherein the flexible element expands radially when compressed axially.

22. The system according to claim 1, wherein the anchoring mechanism comprises a toggle which moves from a collapsed insertion configuration to a deployed anchoring position after insertion into the uterus.

23. The system according to claim 1, wherein the rim includes a protrusion which, when the compression element is in a desired position within the vagina, applies an increased amount of pressure to areas likely to abut the uterine arteries.

24. The system according to claim 1, wherein the anchoring mechanism includes a shaft extending through the compression element and the advancing mechanism comprises a first thread on the shaft and a mating second thread on the compression element so that rotation of the shaft relative to the compression element moves the shaft and the compression element proximally and distally relative to one another.

25. The system according to claim 1, wherein the anchoring mechanism includes forceps.

26. The system according to claim 25, wherein the advancing mechanism comprises a first thread formed on a shaft of the forceps and a mating second thread formed on the compression element.

27. The system according to claim 1, further comprising a handle and wherein the advancing mechanism includes a trigger linked to the compression element to advance the compression element as the trigger is depressed relative to the handle.

28. The system according to claim 27, further comprising a ratchet coupled to the trigger preventing proximal translation of the compression element relative to the handle.

29. The system according to claim 1 28, wherein the ratchet includes an angled plate.

30. A uterine occlusion instrument, comprising:
a shaft including a proximal end which, during use, remains outside the vagina accessible to a user;
a compression element coupled to the shaft, the compression element including a distal rim shaped to engage a target portion of tissue surrounding the cervical opening;
an anchoring mechanism anchoring the compression element in a desired position relative to the cervical opening; and
an advancing mechanism moving the compression element distally relative to the anchoring mechanism to compress the target portion of tissue against an adjacent portion of a uterine wall occluding uterine arteries located therebetween.

31. The uterine occlusion instrument according to claim 30, wherein the compression element is separable from the shaft while the anchoring mechanism maintains the compression element in place compressing the target portion of tissue.

32. The uterine occlusion instrument according to claim 30, wherein the compression element is formed as a cup including an open distal end.

33. The uterine occlusion instrument according to claim 30, wherein the advancing mechanism comprises a vacuum lumen extending into the compression element so that, negative pressure applied to a proximal end of the vacuum lumen is communicated to an interior of the compression element to draw a portion of tissue surrounding the cervical opening proximally thereinto.

34. The uterine occlusion instrument according to claim 30, wherein the anchoring mechanism includes forceps extendable from the shaft to grasp a portion of tissue surrounding the cervical opening.

35. The uterine occlusion instrument according to claim 34, wherein the forceps is biased toward a closed position.

36. The uterine occlusion instrument according to claim 30, wherein the anchoring mechanism includes a thread formed on a distal end of the shaft for anchoring into tissue of a cervical canal.

37. The uterine occlusion instrument according to claim 30, wherein the anchoring mechanism includes an anchoring shaft extending through a cervical canal into the uterus and an anchor extending laterally from a distal end of the anchoring shaft.

38. The uterine occlusion instrument according to claim 37, wherein the anchor comprises first and second anchoring shafts deployable from the anchoring shaft to form a substantially Y-shaped anchor.

39. The uterine occlusion instrument according to claim 30, wherein the anchor includes an inflatable member coupled to a source of inflation fluid.

40. The uterine occlusion instrument according to claim 30, wherein the compression element is formed as a coil.

41. The uterine occlusion instrument according to claim 30, wherein the advancing mechanism includes a first thread formed on the shaft and a second mating thread formed on the compression element so that rotation of the shaft relative to the compression element in a first direction moves the compression element distally relative to the shaft.

42. The uterine occlusion instrument according to claim 30, wherein the advancing mechanism includes a linear advancing mechanism translating the compression element proximally and distally relative to the anchoring mechanism.

43. The uterine occlusion instrument according to claim 30, further comprising a stiffening element inserted into the cervical opening to increase a stiffness of a cervix against which the target portion of tissue and the adjacent portion of uterine wall are compressed.

44. The uterine occlusion instrument according to claim 43, wherein the stiffening element comprises a plug insertable into the cervical opening.

45. The uterine occlusion instrument according to claim 30, wherein the rim extends around substantially all of a perimeter of the cervical opening.

46. The uterine occlusion instrument according to claim 30, wherein the rim includes two wings on opposite sides of an axis of the shaft.

47. The uterine occlusion instrument according to claim 46, wherein each of the wings subtends a range of at least 120E.

48. A method of occluding uterine arteries, comprising:
coupling an anchoring mechanism to tissue adjacent to a cervical opening;
inserting into the vagina a compression element coupled to the anchoring mechanism so that a distal rim thereof engages a target portion of a vaginal formix; and
advancing the compression element distally relative to the anchoring mechanism to compress the target portion of tissue against an adjacent portion of uterine wall occluding uterine arteries compressed therebetween.

49. The method according to claim 48, wherein the rim extends around substantially all of a perimeter of a cervical opening.
